(19) United States
(12) Patent Application Publication
(10) Pub. No.: US 2023/0381204 A1
(43) Pub. Date: Nov. 30, 2023

(54) COMPOSITIONS FOR TREATING INFLAMMATIONS, WOUNDS AND SCARRING

(71) Applicant: KANACARE LTD., Ganei Hadar (IL)

(72) Inventors: Nurit TSUR LEV, Ganey Hadar (IL); Sharon ROZENBLAT, Tel Aviv (IL)

(21) Appl. No.: 18/032,659

(22) PCT Filed: Oct. 19, 2021
(86) PCT No.: PCT/IL2021/051233

§ 371 Date: Apr. 19, 2023

Related U.S. Application Data
(60) Provisional application No. 63/106,378, filed on Oct. 28, 2020.

(51) Int. Cl. A61K 31/00; A61K 36/65; A61K 31/7048; A61K 31/4166; A61P 29/00; A61P 17/02; A61K 9/00

(52) U.S. Cl. CPC A61K 31/658; A61K 36/65; A61K 31/7048; A61K 31/4166; A61P 29/00; A61P 17/02; A61K 9/0014

(57) ABSTRACT

The invention provides synergistic topical formulations comprising a cannabinoid; an extract from Paeonia lactiflora; and optionally allantoin. The invention further provides uses of these formulations in treating certain inflammatory diseases, wounds, scarring and the like. Examples of such diseases or conditions are arthritis, such as rheumatoid arthritis. Other examples are skin diseases or conditions, such as wound and consequent scarring.

### FIELD OF THE INVENTION

The invention relates to synergistic compositions comprising cannabinoids and a plant extract or a combination of terpenes and uses thereof in treating inflammatory disease wounds or scarring or any other disease or condition of skin or connective tissue.

### BACKGROUND OF THE INVENTION

A wound results from damage or disruption to normal anatomical structure and function. This can range from a simple break in the epithelial integrity of the skin to deeper, subcutaneous tissue with damage to other structures such as tendons, muscles, vessels, nerves, parenchymal organs and even bone. Irrespective of the cause and form, wounding damages and disrupts the local tissue environment.

Wound healing is a dynamic, interactive process involving soluble mediators, blood cells, extracellular matrix, and parenchymal cells. The wound repair process can be divided into four (4) temporally and spatially overlapping phases: (1) a coagulation phase, (2) an inflammatory phase, (3) a proliferative phase, and (4) a remodeling phase. Hypertrophic or keloid scars are frequently the sequel of burns, surgical

incisions, injuries and other skin trauma with significant cosmetic and functional consequences, especially when it is in the face and across joints. There are limited therapeutic options for the reduction of scarring following these injuries beyond promotion of re-epithelialization and control of inflammation. Mechanical manipulation to reduce tension in surgical incisions can improve scar outcome, but are not practical for burn scars or large post traumatic injuries.

Administration of pharmaceutical agents by intradermal injection is a challenging treatment modality, which is painful, and requires a health care professional. Comparing to intradermal injection, topical application has many advantages such as ease of handling, the localized delivery of product and the reduced effect of first pass metabolism etc. Furthermore, a suitable concentration of therapeutic agent can be applied topically without a risk of systemic effects.

Experiments in tissue repair and treatment of existing pathological scars and symptoms associated with such scarring notwithstanding, clinical success has remained elusive, and the various biological and molecular mechanisms responsible for normal tissue repair and pathological scarring have yet to be fully elucidated.

To date there is no approved pharmaceutical product in the US or the EU indicated for the reduction, improvement, or prevention of cutaneous scarring in humans. Thus, unfortunately, scar improvement and prevention of scar formation still remains an area of clearly unmet medical need.

Further there is still an unmet need for the development of compositions for wound healing and scarring.

### SUMMARY OF THE INVENTION

The invention is based on the unexpected observation that a formulation comprising cannabidiol (CBD) and/or cannabigerol (CBG) in combination with: a plant extract of Paeonia lactiflora; and/or a specific combination of terpenes, provided a formulation with a synergistic anti-inflammatory effect, synergistic wound healing effect and/or synergistic scarring effect. In some embodiments, the composition that comprises CBD is a composition that comprises either pure CBD or more than 81, 83, 85, 87, 89, 90, 92, 94, 96, 98, 99 wt % CBD. Such a composition may contain small amounts of CBG, for example in an amount which is more than about 1%, 2%, 3%, 4%, 5%, 6% , 7%, 8%, 9% 10 wt % or more of the total weight of the CBD. In some embodiments, the composition is devoid of THC. It is to be understood that the CBD or CBG in the composition are diluted in the formulation and that the final concentration thereof in the formulation of the invention is less than 10, 9, 8, 7, 6, 5, 4, 3, 2, 1, 0.1, 0.01, 0.001% wt.

In some embodiments, the composition that comprises CBG is a composition that comprises pure CBG or more than 81, 83, 85, 87, 89, 90, 92, 94, 96, 98, 99 wt % CBG. In some embodiments, the composition is devoid of THC. In particular, it was shown that secretion of IL-1α, IL-6 and TNFα, which are associated with different inflammatory disorders including wound healing and formation of scars, e.g. keloid scars, was significantly reduced upon contact with the formulation comprising a plant extract of Paeonia lactiflora and a composition that comprises CBD with or without allantoin of the invention. Secretion of IL-1α and TNFα, was significantly reduced upon contact with the formulation comprising a plant extract of Paeonia lactiflora and a composition that comprises CBG.

According to one aspect, the invention provides a topical synergistic formulation comprising a cannabinoid and a plant extract of Paeonia lactiflora, wherein the cannabinoid is selected from cannabidiol (CBD) or a composition comprising CBD (as hereindefined) and/or cannabigerol (CBG) or a composition comprising CBG (as hereindefined) and the combination of thereof. In some embodiments, the formulation further comprises allantoin.

According to some embodiments, the composition comprises CBD and/or CBG further comprises a combination of terpenes. According to certain embodiments, the combination of terpenes comprises from about 15 to about 35 wt % of myrcene; from about to about 25 wt % of terpinolene; from about 10 to about 25 wt % of limonene; from about to about 15 wt % of ocimene; from about 2 to about 10 wt % of β-pinen and β-caryophyllene; and from about 1 to about 7 wt % of α-pinene and Y-terpinene. According to some embodiments, the weight ratio between the cannabinoid and the combination of terpenes is from 1:1000 to 1000:1.

According to other embodiments, the formulation comprises CBD or CBG and a plant extract of Paeonia lactiflora. According to certain embodiments, the extract of Paeonia lactiflora is a root extract. Such an extract comprises, according to some embodiments, from 2 to 10 wt % of paeoniflorin. According to some embodiments, the weight ratio between the cannabinoid and the Paeonia lactiflora extract is from 1:100,000 to 1:100 or from 1:100 to 100:1 or is less than 1:90, 1:80, 1:70, 1:60, 1:50, 1:40; 1:30; 1:20, 1:10 or 1:1.

According to some embodiments, the formulation comprises from 0.1 to 10 wt % of the cannabinoid and from 0.01 to 10 wt % of the extract and/or the combination of terpenes.

According to some embodiments, there is provided a topical formulation comprising a cannabinoid or a composition comprising cannabinoides; a plant extract of Paeonia lactiflora; optionally allantoin; and a carrier, wherein the cannabinoid is selected from cannabidiol (CBD) and cannabigerol (CBG). In some embodiments, the composition comprising cannbinoid comprises 80, 81, 82, 83, 84, 85, 86, 87, 88, 89, 90, 91, 92, 93, 94, 96, 97, 98, 99 wt % or more CBD. In some embodiments, the composition comprising cannabinoides further comprises terpens in an amount of less than 5%, 4%, 3%, 2% or 1% or less. In some embodiments, the weight ratio between the cannabinoid and the combination of terpenes is from 1:1000 to 1:50. In some embodiments, the formulation comprising composition comprising CBD in a form of an oil comprising CBD in an amount of 80, 81, 82, 83, 84, 85, 86, 87, 88, 89, 90, 91, 92, 93, 94, 95, 96, 97, 98, 99 wt % or more CBD; the formulation further comprises plant extract of Paeonia lactiflora; and optionally allantoin.

In some embodiments, formulation comprises CBG or oil comprising CBG in an amount of 80, 81, 82, 83, 84, 85, 86, 87, 88, 89, 90, 91, 92, 93, 94, 95, 96, 97, 98, 99% wt % or more CBG; the plant extract of Paeonia lactiflora; and allantoin. It is to be understood that the CBD or CBG in the composition are diluted in the formulation and that the final concentration thereof in the formulation of the invention is less than 10, 9, 8, 7, 6, 5, 4, 3, 2, 1, 0.1, 0.01, 0.001% wt.

In some embodiments, the extract of Paeonia lactiflora is a root extract.

In some embodiments, Paeonia lactiflora extract comprises from 2 to 10 wt % of paeoniflorin.

In some embodiments, the weight ratio between the cannabinoid and Paeonia lactiflora extract is from 1:100 to 100:1. In some embodiments, the weight ratio between the cannabinoid and the extract is from 1:1 to 1:100.

In some embodiments, the formulation comprises from 0.1 to 10 wt % of CBD or of a composition comprising CBD in an amount of 80, 81, 82, 83, 84, 85, 86, 87, 88, 89, 90, 91, 92, 93, 94, 95, 96, 97, 98, 99 wt % or more; and from 0.01 to 10 wt % of Paeonia lactiflora extract.

In some embodiments, the composition comprising CBD further comprises CBG in a ratio of between 1:1 to 1:1000 of the CBD.

In some embodiments, the formulation further comprises allantoin In some embodiments, the formulation comprises from 0.1 to 10 wt % of

cannabinoid or of a composition comprising CBG in an amount of 80, 81, 82, 83, 84, 85, 86, 87, 88, 89, 90, 91, 92, 93, 94, 95, 96, 97, 98, 99 wt % or more; from 0.01 to 10 wt % of Paeonia lactiflora extract.

In some embodiments, the composition comprising CBG further comprises CBD in a ratio of between 1:1 to 1:100 of the CBG.

In some embodiments, the formulation further comprises allantoin

In some embodiments, the formulation provides a synergistic anti-inflammatory effect.

In some embodiments, the formulation provides a synergistic wound-healing effect.

In some embodiments, the synergistic wound-healing effect comprises a synergistic anti-scaring effect.

In some embodiments, formulation is a pharmaceutical formulation comprising a pharmaceutically acceptable carrier.

In some embodiments, the formulation described herein is for use in treating a disease selected from connective tissue disease and a skin disease, disorder or condition. In some embodiments, the skin disease, disorder or condition comprises wound.

In some embodiments, treating wound comprises improving wound healing.

In some embodiments, treating the disease, disorder or condition comprises treating a scar or preventing scar formation.

In some embodiments, treating a scar comprises reducing scar collagen abundance, scar width, and scar tissue contracture and decreasing scar formation, preventing scar formation and reducing scar. In some embodiments, the scar is keloid or hypertrophic scar.

In some embodiments, connective tissue disease comprises an arthritis. In some embodiments, the arthritis is a rheumatoid arthritis. In some embodiments, there is provided a method for treating scar or preventing scar formation in a subject in need thereof comprising topically administering the formulation described herein.

According to some embodiments, the formulation is a dermal composition comprising a pharmaceutically acceptable carrier.

According to some embodiments, the formulation is a cosmetic composition comprising a cosmetic acceptable carrier.

### BRIEF DESCRIPTION OF DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A, 1B, 1C and 1D show evaluation in LPS-induced cytokine secretion model in control samples in RAW 264.7 cells (Mouse monocyte macrophage) obtained originally from ECACC: FIG. 1A shows the results of MTT test; FIG. 1B shows IL-1α secretion inhibition; FIG. 1C shows TNF-α secretion inhibition and FIG. 1D shows IL-6 secretion inhibition. Cell culturing and treatments was performed under sterile conditions. Cell viability was determined by MTT and cytokine secretion by ELISA. Mean±SEM; n=3. */#p<0.05 for differences from the Naïve or LPS control groups, respectively. Synergy is indicated by $.

FIGS. 2A, 2B, 2C, 2D: synergy anti-inflammatory evaluation of CBD with Paeonia lactiflora extract in LPS-induced cytokine secretion model in RAW cells: The cells were treated w/o or with the individual Tested Samples or combinations thereof. Cell viability was determined by MTT (FIG. 2A) and cytokine secretion by ELISA (FIG. 2B, 2C, 2D). Mean±SEM; n=3. */#p<0.05 for differences from the Naïve or LPS control groups, respectively. Synergy is indicated by $. CBD and Paeonia lactiflora extract (may be also termed here TI6, 6_1, sample 6 or herbal extract or P. lactiflora) assessed in concentration of 1:1000 μg/ml.

FIG. 2A shows the results of MTT test; FIG. 2B shows the IL-1α secretion inhibition; FIG. 2C shows TNF-α secretion inhibition and FIG. 2D shows IL-6 secretion inhibition.

FIGS. 3A, 3B and 3C: synergy anti-inflammatory evaluation in LPS-induced cytokine secretion model in RAW cells: The cells were treated w/o or with the individual Tested Samples or combinations thereof, as described above. Cell viability was determined by MTT (FIG. 3A) and cytokine secretion by ELISA (FIG. 3B, 3C). Mean±SEM; n=3. */#p<0.05 for differences from the Naïve or LPS control groups, respectively. Synergy is indicated by $. CBG and Paeonia lactiflora extract (may be also termed here TI6, 6_1, herbal extract or P. lactiflora) assessed at 1000 μg/ml FIG. 3A shows the results of MTT test; FIG. 3B shows the IL-1α secretion inhibition; FIG. 3C shows TNF-α secretion inhibition.

FIGS. 4A, 4B, 4C, 4D and 4E demonstrate the anti-inflammatory synergistic activity (TNF alpha inhibition) in RAW cells in LPS-induced inflammatory mode. The cells were treated w/o or with the individual Tested Samples or combinations, as described above. Synergistic anti-inflammatory activity was shown between:
Cannabis oils (1, 2) and Paeonia lactiflora extract (may be also termed here herbal extract, P. lactiflora or (3))
Cannabis oil (1) and terpenes mixture (may be also termed here 4)
Cannabis oil (2), Paeonia lactiflora extract (may be also termed here herbal extract,
P. lactiflora or (3) and Allantoin (5)

Naive cells, vehicle treated cells, Stimulated Control and Stimulated Vehicle Control served as controls. Dexamethasone served as positive control for anti-inflammatory assay. A Blank control group was included in the assay. FIG. 4A describes the controls used in the study and the stimulation effect of LPS.

CBD oil 1 (1:1,000,000 dilution) exhibited 13.7% TNF alpha secretion inhibition alone, Paeonia lactiflora root extract (40 μg/ml) showed no activity alone, but together they exhibited synergistic anti-inflammatory activity of 46.7% inhibition (FIG. 4B). When CBD oil 1 at the same concentration measured together with terpene mixture (16.3% inhibition), combination showed synergistic anti-inflammatory activity of 60.6% TNF alpha secretion inhibition (FIG. 4C). CBD oil 2 (1:1,000,000 dilution) inhibited TNF alpha by 17.2% alone, Paeonia lactiflora herbal extract (40 μg/ml) showed no inhibition activity alone, but together they exhibited synergistic anti-inflammatory activity of 42.4% TNF alpha secretion inhibition (FIG. 4D). CBD oil 2 (1:500,000 dilution) exhibited 42.9% TNF alpha inhibition alone, Paeonia lactiflora herbal extract (40 μg/ml) and allantoin (2 μg/ml) showed no activity alone, but the three compounds together exhibited synergistic anti-inflammatory activity of 56.7% TNF alpha inhibition (FIG. 4E).

FIGS. 5A, 5B, 5C, 5D, 5E, 5F and 5G: Synergy evaluation in scratch assay model: HaCaT keratinocyte cells were treated with the indicated tested samples for 48 hr. First, to ascertain the non-toxic range, MTT assay was employed (data not shown). Next, the scratch assay was employed on the test items alone and their combinations to test the impact of the Test items and their combinations on dermal wounds. CBD oil 1 and 2 and Paeonia lactiflora herbal extract have been tested alone and in combination with or without Allantoin. Mean±SEM; n=3. *p<0.05 for differences from the Naïve, respectively. Synergy is indicated by $. FIG. 5A presents photos of the cells treated as follows: Upper row from left to right: Control 481.8 micronCBD oil 1 (1:500,000), CBD oil 1 (1:1,000,000); Middle row from left to right: CBD oil 2 (1:500,000); Paeonia lactiflora 20 μg/ml (may also termed here as herbal extract or P. lactiflora); Allantoin(40 m/ml); Bottom row from left to right: CBD oil 1(1:500,000)+Paeonia lactiflora extract (P. lactiflora), 20 μg/ml; CBD oil 1 (1:1,000,000)+Paoenia lactiflora extract (P. lactiflora), 20 μg/ml); and CBD oil 2 (1:1,000,000). FIG. 5B presents photo of the cells treated with CBD oil 2 (1:500,000)+Paeonia lactiflora extract (P. lactiflora), 20 μg/ml=153 micron. This combination demonstrated 68% closure. FIG. 5C presents photo of the cells treated with CBD oil 2 (1:1,000,000)+Paeonia lactiflroa extract (P. lactiflora), 20 μg/ml=129.7 micron. This combination demonstrated 73% closure. FIG. 5D presents photo of the cells treated with CBD oil 1 (1:1,000,000)+Paeonia lactiflora extract (P. lactiflora), 20 μg/ml+Allantoin (40 μg/ml)−106.4 micron which showed 77.9% closure. FIG. 5E shows graph with bars presenting the closure of the “wound” (“wound length”) showed in FIG. 5A-D with the different treatments. FIGS. 5F and 5G show the significant synergism of CBD oil 2 at different dilutions: (1:500,000) and (1:1,000,000), respectively with Paeonia lactiflora extract (P. lactiflora)(20 μg/ml) (also termed here herbal extract). Synergy is indicated by $.

### DETAILED DESCRIPTION OF THE INVENTION

The invention is based on an unexpected observation that a combination of cannabinoids with an extract of Paeonia lactiflora optionally with allantoin provides a synergistic anti-inflammatory, wound healing or scar prevention or healing effect.

In some aspects, the invention provides a topical synergistic formulation comprising a cannabinoid or an oil comprising cannabinoid and (i) a plant extract of Paeonia lactiflora and/or (ii) a combination of terpenes, and a carrier and/or excipients, wherein the combination of terpenes comprises one or more of myrcene, terpinolene, limonene, ocimene, β-pinen, β-caryophyllene, and α-pinene terpenes. In some embodiments, formulation further comprises allantoin. According to one embodiment, the cannabinoid is selected from cannabidiol (CBD) and cannabigerol (CBG). In some embodiments, the CBD composition comprises at least 80, 81, 82, 83, 84, 85, 86, 87, 88, 89, 90, 91, 92 93, 94, 95, 96, 97, 98, 99 wt % CBD and is in a form of oil. In some embodiments, CBG is also included within the CBD oil composition is in a weight ratio of between 1:1 to 1:100, 1:200, 1:250, 1:500, 1:750, 1:1000 or even 1:2000 of the CBD.

In some embodiments, the CBG composition comprises at least 80, 81, 82, 83, 84, 85, 86, 87, 88, 89, 90, 91, 92 93, 94, 95, 96, 97, 98, 99 wt % CBG. In some embodiments, CBD is also included within the CBG composition is in a weight ratio of between 1:1 to 1:100 1:200, 1:250, 1:500, 1:750, 1:1000 or even 1:2000 of the CBG. In some embodiments, the CBG composition is in a form of oil. In one aspect, the invention provides a topical formulation comprising (i) a cannabinoid or a composition comprising cannaboid and (ii) a plant extract of Paeonia lactiflora and a carrier and/or excipient, wherein the cannabinoid is consisting essentially of cannabidiol (CBD) and/or cannabigerol (CBG) or combination thereof, wherein the topical synergistic formulation may further comprises allantoin. It is to be understood that the CBD or CBG in the composition are diluted in the formulation and that the final concentration thereof in the formulation of the invention is less than 10, 9, 8, 7, 6, 5, 4, 3, 2, 1, 0.1, 0.01, 0.001% wt.

According to some embodiments, the formulation is synergistic formulation. Thus, according to some aspects, the invention provides a topical synergistic formulation comprising (i) a cannabinoid and (ii) a plant extract of Paeonia lactiflora and/or a combination of terpenes, and a carrier and/or excipient, wherein the cannabinoid is selected from cannabidiol (CBD) and/or cannabigerol (CBG), and/or the combination of terpenes comprises at least two or more of the following terpenes: bisabolol, linalool myrcene, terpinolene, limonene, ocimene, β-pinen, β-caryophyllene and α-pinene terpenes. According to some embodiments, the synergistic formulation is a synergistic anti-inflammatory composition, a synergistic wound healing mixture and/or a synergistic scaring preventing or scaring healing formulation.

As used herein, the term “synergistic” refers to a combination of compounds which, when taken together, is more effective than the additive effects of the individual therapies. A synergistic effect of a combination of therapies (e.g., a combination of therapeutic agents) may permit to use of lower dosages of one or more of the therapeutic agent(s) and/or less frequent administration of the agent(s) to a subject with a disease or disorder, e.g., a proliferative disorder. The ability to utilize lower the dosage of one or more therapeutic agents and/or to administer the therapeutic agent less frequently reduces the toxicity associated with the administration of the agent to a subject without reducing the efficacy of the therapy in the treatment of a disease or disorder. In addition, a synergistic effect can result in improved efficacy of agents in the prevention, management or treatment of a disease or disorder, e.g. a proliferative disorder. Finally, a synergistic effect of a combination of therapies may avoid or reduce adverse or unwanted side effects associated with the use of either therapeutic agent alone.

The terms “cannabidiol” and “CBD” are interchangeably used herein and refer to a non-psychotropic cannabinoid having the structure as described in Formula I below, salt or derivatives thereof, such as Δ4-cannabidiol, Δ5-cannabidiol, Δ6-cannabidiol, Δ1,7-cannabidiol, Δ1-cannabidiol, Δ2-cannabidiol, Δ3-cannabidiol.

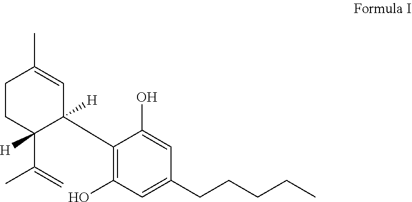 [SMILES: C=C(C)[C@@H]1CCC(C)=C[C@H]1c1c(O)cc(CCCCC)cc1O]

As used herein, the term “CBG” refers to cannabigerol and has the structure as described in Formula II:

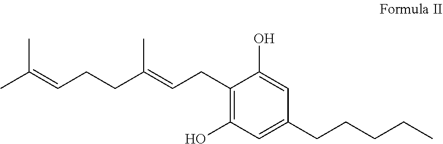 [SMILES: CCCCCc1cc(O)c(C/C=C(\C)CCC=C(C)C)c(O)c1]

The term “terpene” or “terpenes” are used herein interchangeably and refer to terpenes naturally present in cannabis plant. The terpenes include, but not limited to linalool, carophyllene oxide, nerolidol, phytol, trans-(3-ocimene, α-trans-caryophyllene, α-humulene, linalyl acetate, borneol, myrcene, terpinolene, limonene, ocimene, β-pinen, β-caryophyllene and α-pinene and any combination thereof.

According to one embodiment, the cannabinoid is cannabidiol (CBD). According to another embodiment, the cannabinoid is Cannabigerol (CBG).

According to one embodiment, the invention provides a topical synergistic formulation comprising CBD and a combination of terpenes, wherein the combination comprises at least two or more of the following terpenes: bisabolol, linalool, myrcene, terpinolene, limonene, ocimene, β-pinen, β-caryophyllene and α-pinene terpenes.

According to one embodiment, the invention provides a topical synergistic formulation comprising CBG and a combination of terpenes, wherein the combination comprises at least two or more of the following terpenes: bisabolol, linalool myrcene, terpinolene, limonene, ocimene, β-pinen, β-caryophyllene and α-pinene terpenes.

According to some embodiments, the formulation comprises from 0.01 to 10 wt % cannabinoid. According to some embodiments, the formulation comprises from 0.05 to 8 wt %, cannabinoid. According to other embodiments, the formulation comprises from 0.1 to 5 wt %, from 0.2 to 1.5 wt %, from 0.3 to 1.2 wt %, from 0.5 wt % to 1 wt %, from 0.6 wt% to 0.8 wt %, from 0.1 to 1 wt %, or from 0.2 to 0.8 wt % cannabinoid.

According to some embodiments, the invention provides a topical synergistic formulation comprising CBD or an oil comprising CBD in an amount of 80, 81, 82, 83, 84, 85, 86, 87, 88, 89, 90, 91, 92, 93, 94, 95, 96, 97, 98, 99 wt % or more and an extract of Paeonia lactiflora. In some embodiments, the oil comprising CBD further includes CBG in a ratio of more than 1:1, 1:2, 1:3, 1:4, 1:5, 1:10, 1:20, 1:30, 1:40, 1:50, 1:60, 1:70:1:80, 1:90, 1:100, 1:125, 1:150, 1:200, 1:000 or more of CBD/CBG.

According to one embodiment, the invention provides a topical synergistic formulation comprising CBG or of oil comprising CBG in an amount of 80, 81, 82, 83, 84, 86, 87, 88, 89, 90, 91, 92, 93, 94, 95, 96, 97, 98, 99 wt % or more and an extract of Paeonia lactiflora. According to some embodiments, the extract is a root extract. In some embodiments, the oil comprising CBG further includes CBD in a ratio of more than 1:1, 1:2, 1:3, 1:4, 1:5, 1:10, 1:20, 1:30, 1:40, 1:50, 1:60, 1:70:1:80, 1:90, 1:100, 1:125, 1:150, 1:200, 1:1000 or more of CBG/CBD.

In some embodiments, there is provided a formulation comprising a composition comprising cannbinoides such as CBD and/or CBG in a form of an oil that further comprises a mixture of terpens is an amount of less than 0.01, 0.05, 0.1, 0.5, 1, 1.5, 2, 2.5, 3, 3.5, 4, 4.5, 5, or 10 wt % of the oil composition. The formulation further comprising Paeonia lactiflora extract, wherein the ratio between the main cannabinoid and Paeonia lactiflora extract is more than 1:100, 1:80, 1:60, 1:40 or 1:20, 1:10 or 1:1.

The term “extract” as used herein refers to a product prepared by extraction by physical means (e.g. by comminuting, pressing, heating, pulsed electric field assisted treatments, shear treatments and pressure wave treatments), by chemical means (e.g. by treatment with an acid, a base, a solvent) and/or by biochemical means (e.g. by treatment with hydrolytic enzymes, microorganisms). The term refers to a liquid substance obtained through extraction from a given substance, or to a concentrate or essence which is free of, or substantially free of solvent. The term extract may be a single extract obtained from a particular extraction step or series of extraction steps or the extract also may be a combination of extracts obtained from separate extraction steps or separate feedstocks. Such combined extracts are thus also encompassed by the term “extract.” Any method of extraction and any solvent is encompassed. The extract may be obtained from any part of the plant e.g. from leaves, flowers, stems, roots, fruits and seeds. The extract may be aqueous or oily. Commonly used solvents are water, ethanol, ethyl acetate, CO₂, methanol, acetone, and acetic acid. In particular, the term “extract” refers to a liquid or semi-solid or resinous substance obtained through extraction from plants defined in the present application. In some embodiments, the term refers also to a compound purified from the extract. According to some embodiments, the term “extract” has the meaning of a mixture or combination of two or more extracts. The term refers also to a dried product of the extract.

According to some embodiments, the extract is a dry extract.

According to some embodiments, the extract of Paeonia lactiflora comprises from 1 to 15 wt % of paeoniflorin. According to some embodiments, the extract comprises from 2 to 12, from 3 to 10, from 4 to 8, from 2 to 6 or from 3 to 5 wt % of paeoniflorin or about 4 wt % paeoniflorin.

According to some embodiments, the weight ratio between the cannabinoid and the Paeonia lactiflora extract is from 1:100,000 to 1:100. According to one embodiment, the weight ratio between the cannabinoid and the extract is from 1:50,000 to 1:200, from 1:20,000 to 1:300. From 1:10,000 to 1:500, from 1:7,500 to 1:750, from 1:5,000 to 1:1000, from 1:3000 to 1:2000 or from 1:100 to 1:1

According to one embodiment, the weight ratio between the cannabinoid and the Paeonia lactiflora extract is from 1:100 to 100:1, from 1:90 to 90:1, from 1:80 to 80:1, from 1:70 to 70:1, from 1:60 to 60:1, from 1:50 to 50:1, from 1:40 to 40:1, from 1:30 to 30:1, from 1:20 to 20:1 or from 1:10 to 10:1. According to another embodiment, the weight ratio between the cannabinoid and the extract is from 1:9 to 9:1, from 1:8 to 8:1, from 1:7 to 7:1, from 1:6 to 6:1, from 1:5 to 5:1, from 1:4 to 4:1, from 1:3 to 3:1, from 1:2 to 2:1 or about 1:1. According to some embodiments, the composition comprises from 0.1 to 10 wt % of the cannabinoid and from 0.01 to 10 wt % of the combination of the extract. According to some embodiments, the composition comprises from 0.5 to 8 wt %, from 1 to 6 wt % or from 2 to 5 wt % of the cannabinoid. According to other embodiments, the composition comprises from 0.1 to 2 wt %, from 0.2 to 1.5 wt %, from 0.3 to 1.2 wt %, from 0.5 wt % to 1 wt %, from wt % to 0.8 wt %, from 0.1 to 1 wt %, or from 0.2 to 0.8 wt % of the cannabinoid. According to other embodiments, the composition comprises from 0.5 to 8 wt %, from 1 to 6 wt % or from 2 to 5 wt % of the extract. According to some embodiments, the composition comprises from 0.2 to 8 wt %, from 0.5 to 6 wt %, from 0.8 to 5 wt %, from 1 to 4 wt %, from 2 to 4 wt%, from 0.1 to 2 wt %, from 0.2 to 1.8 wt %, from 0.5 to 1.5 wt %, or from 0.8 to 1.2 wt % of the extract. According to some embodiments, the composition comprises from 0.01 to 2 wt %, from 0.02 to 1.8 wt %, from 0.03 to 1.5 wt %, from 0.05 to 1.2 from 0.08 to 1 wt %, from 0.1 to 1 wt %, from 0.2 to 0.8, rom 0.5 wt % to 1 wt %, from 0.01 to 1 wt %, from 0.03 to 0.9 wt %, from 0.05 to 0.5 wt %, of from 0.1 to 0.3 wt % of the extract.

According to some embodiments, the weight ratio between CBD and the Paeonia lactiflora extract is from 1:50,000 to 1:1,000, from 1:25,000 to 1:2000, from 1:15,000 to 1:3,000, from 1:10,000 to 1:5,000., or from 1:100 to 1:1.

According to other embodiment, the weight ratio between CBG and the Paeonia lactiflora extract is from 1:10,000 to 1:100, from 1:5,000 to 1:200, from 1:3000 to 1:300, from 1:2000 to 1:300 or from 1:100 to 1:1.

In some embodiments, the formulation further comprises between 0.1-10 wt % allantoin. In some embodiments, the formulation further comprises between 0.1-5 wt % allantoin. In some embodiments, the formulation further comprises between 0.1-3 wt % allantoin. In some embodiments, the formulation further comprises between 0.2-0.8 wt % allantoin. In some embodiments, the formulation further comprises between 0.4-0.6 wt % allantoin. In some embodiments, the formulation further comprises about 0.5 wt % allantoin.

According to any one of the above embodiments, the formulation of the present invention provides a synergistic anti-inflammatory effect. According to some embodiments, the anti-inflammatory effect comprises wound-healing effect. According to some embodiments, the formulation of the invention provides a synergistic wound-healing effect. According to other embodiments, the synergistic wound-healing effect comprises a synergistic anti-scarring effect.

According to other embodiments, the synergistic anti-inflammatory effect comprises anti-arthritic effect, such as an effect against rheumatoid arthritis.

According to some embodiments, the carrier is a carrier used in topical compositions as known in the art.

According to any one of the above embodiments, the formulation of the invention is a pharmaceutical formulation, and the carrier and/or excipient is a pharmaceutically acceptable carrier and/or excipient. According to any one of the above embodiments, the formulation of the invention is a cosmetic composition, and the carrier and/or excipient is a cosmetically acceptable carrier and/or excipient.

According to any one of the above embodiments, the formulation of the invention is a dermatological formulation, and the carrier and/or excipient is a dermatically acceptable carrier and/or excipient.

Thus, according to some embodiments, the present invention provides a topical synergistic pharmaceutical composition comprising a cannabinoid and (i) a plant extract of Paeonia lactiflora and/or (ii) a combination of terpenes, and may further contain allantoin and a pharmaceutically acceptable carrier and/or excipient, wherein the cannabinoid is selected from cannabidiol (CBD) and cannabigerol (CBG), or an oil composition that contains at least 80, 81, 82, 83, 84, 85, 86, 87, 88, 89, 90, 91, 92 93, 94, 95, 96, 97, 98, 99 wt % CBD or CBG, respectively. In some embodiments, CBG is also included within the CBD oil composition and is in a weight ratio of between 1:1 to 1:100 or even 1:200 1:1000of the CBD.

In some embodiments, CBD is also included within the oil CBG composition is in a weight ratio of between 1:1 to 1:100 or even 1:200 1:1000 of the CBG.

The term “pharmaceutical composition” or “pharmaceutical formulation” as used herein refers to a composition comprising the active agents as disclosed herein formulated together with one or more pharmaceutically acceptable carriers and/or excipients.

Formulations of the pharmaceutical composition may be adjusted according to applications. In particular, the pharmaceutical composition may be formulated using a method known in the art so as to provide a rapid, continuous or delayed release of the active ingredient after administration to mammals. For example, the formulation may be any one selected from among plasters, powders, ointments, liquids and solutions, aerosols, extracts, elixirs, fluidextracts, emulsions, suspensions, decoctions, infusions, suppositories, injections, spirits, creams, troches, and tinctures.

The term “topical administration” or “topical application” refers to directly layering or spreading upon epidermal tissue, especially outer skin or membrane, including the skin or membrane of the oral, anorectal or vaginal cavities.

The term “topical composition” or “topical formulation” refers to a material that comprises the active pharmaceutical ingredients (API), a dermatologically acceptable carrier which may be pharmaceutically acceptable carrier and is intended for administration to an animal or human subject and is applied to the surface of the skin, in contrast to materials that are taken orally or via intravenous (subdermal) injection. A topical composition is generally intended to have its intended effect at the site of application or in proximity to it. Topical compositions as provided herein are typically administered for the purpose of alleviation of symptoms associated with a dermatological disease or condition, treatment of a dermatological disease or condition, or prevention of a dermatological disease or condition as well as alleviation of the symptoms and/or treatment of diseases or conditions of tissues located in proximity to skin like tendons, ligaments, joints and any other tissue. The compositions of the present invention may be in any of the galenical forms normally used for topical administration, in particular in the form of solutions, lotions, gels, emulsions of liquid or semi-liquid consistency of the milk type, suspensions or emulsions of soft, semi-liquid or solid consistency, of the cream or ointment type, or else micro-emulsions, microcapsules, microparticles or vesicular dispersions of ionic and/or nonionic type.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions, solid carriers or excipients such as, for example, lactose, starch or talcum or liquid carriers such as, for example, water, fatty oils or liquid paraffins.

The carrier of the invention is adapted to the topical mode of administration and therefore being a topically/cosmetically/dermatologically acceptable carrier. The terms “cosmetically acceptable carrier”, “dermatologically acceptable carrier” and “carrier for topical compositions” are used herein interchangeably and refer to a carrier, excipient or diluent that is suitable for use in contact with the skin without undue toxicity, incompatibility, instability, irritation, allergic response, and the like. It is appreciated that all carriers, excipients and diluents which can be added to the cosmetic composition of the present invention are approved for human and animal for cosmetic use.

Suitable carriers, excipients, or diluents include, but are not limited to, starch, glucose, lactose, sucrose, gelatin, silica dioxide, sodium stearate, glycol monostearate, talc, sodium chloride, dried skim milk, glycerol, propylene glycol and the like. Suitable waxes include but not limited to beeswax, lanolin, candelilla, soy, and carnauba wax. According to some embodiments, the carrier is liquid or semi-liquid silicon.

Suitable further excipients include, but are not limited to, viscosity modifiers, emulsifiers, film-forming agents, foaming agents, colorants, preservatives, fragrance agents, solvents, electrolytes, pH adjusting agents, and combinations thereof.

Suitable viscosity modifier includes, but is not limited to, carboxymethyl cellulose, methyl cellulose, ethyl cellulose, hydroxyethyl cellulose, hydroxypropyl cellulose, hydroxypropylmethyl cellulose, silicates, and combinations thereof.

Suitable emulsifier includes, but is not limited to, lecithin, hydrogenated lecithin, glycerol, hydrogenated castor oil, cetearyl alcohol, behenyl alcohol, butylene glycol, propylene glycol, and combinations thereof.

Suitable colorant includes, but is not limited to, inorganic pigments, organic pigments, and combinations thereof.

Suitable inorganic pigment includes, but is not limited to, rutile titanium dioxide, anatase titanium dioxide, zinc oxide, zirconium oxide, iron oxides chromium oxide, chromium hydroxide, bismuth oxy chloride, manganese violet, cerium oxide, ultramarine blue, carmine, and combinations thereof.

Suitable organic pigment includes, but is not limited to, dihydroxyacetone (DHA), erythrulose, carbonyl derivatives, and combinations thereof

Suitable preservative includes, but is not limited to, butyl paraben, methyl paraben, propyl paraben, propionate salts, quaternary ammonium compounds, butyl benzoate, benzyl alcohol, benzoic acid, and combinations thereof.

Suitable fragrance agent includes, but is not limited to, essential oil, plant extract, and combinations thereof.

Suitable solvent includes, but is not limited to, water, ethanol, methanol, propanol, hexanol, propylene glycol, pentylene glycol, hexylene glycol, 1, 2-octanediol, sorbitol, glycerol, ethyl acetate, and combinations thereof.

Suitable electrolyte includes, but is not limited to, sodium chloride.

Suitable pH adjusting agent includes, but is not limited to, organic and mineral acids or bases.

According to additional embodiments, the formulation may further comprise vitamins. Suitable vitamins include e.g. vitamin A and derivatives, including retinoic acid, retinyl aldehyde, tretinoin (Retin A®), retinyl palmitate, adapalene, and beta-carotene; vitamin B (panthenol, provitamin B5, panthenic acid, vitamin B complex factor); vitamin C (ascorbic acid and salts thereof) and derivatives such as ascorbyl palmitate; vitamin D including calcipotriene (a vitamin D3 analog) vitamin E including its individual constituents alpha-, beta-, gamma-, delta-tocopherol and cotrienols and mixtures thereof and vitamin E derivatives including vitamin E palmitate, vitamin F linolate and vitamin E acetate; vitamin K and derivatives; vitamin Q (ubiquinone) and combinations thereof. According to any one of the above embodiments, the composition of the present invention is for use in treating a disease selected from a connective tissue disease and a skin disease, disorder or condition. All the above definitions and embodiments apply herein as well.

According to additional embodiments, the formulation may further comprise allantoin and zinc derivates.

According to one embodiment, the present invention provides a topical synergistic composition comprising CBG or CBD and a combination of terpenes, and a carrier and/or excipients, wherein the combination comprises myrcene, terpinolene, limonene, ocimene, β-pinen, β-caryophyllene and α-pinene terpenes for use in treating a skin disease, disorder or condition.

According to another embodiment, the invention provides a topical synergistic formulation comprising CBG or CBD or a composition comprising thereof as defined herein and a combination of terpenes and a carrier and/or excipients, wherein the combination comprises myrcene, terpinolene, limonene, ocimene, β-pinen, β-caryophyllene and α-pinene terpenes for use in treating a connective tissue disease. According to yet another embodiments, the present invention provides a topical synergistic formulation comprising CBG or CBD or a composition comprising thereof as defined herein and an extract of Paeonia lactiflora, and a carrier and/or excipients, for use in treating a skin disease, disorder or condition. According to yet another embodiment, the invention provides a topical synergistic formulation comprising CBG or CBD or a composition comprising thereof as defined herein and an extract of Paeonia lactiflora, and a carrier and/or excipients, for use in treating a connective tissue disease. According to some embodiments, the extract is a root extract. According to an embodiments of the invention, the formulation further comprises allantoin.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting the development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

The term “inflammation” as used herein refers to the physiologic process by which vascularized tissues respond to injury. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses. The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents. The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

According to some embodiments, the skin disease, disorder or condition is selected from wounds, atopic dermatitis, allergic contact dermatitis, irritant contact dermatitis, radiation dermatitis, seborrheic dermatitis, psoriasis, sunburn, diabetic ulcers, pressure ulcers, and stasis ulcers. Dermatological conditions can also arise from irritation and/or pain following laser or chemical resurfacing, dermabrasion therapy, cuts, burns, and abrasions.

According to some embodiments, the skin disease, disorder or condition comprises wound. According to some embodiments, the composition of the present invention is for use in improving wound healing. According to other embodiments, treating the disease, disorder or condition comprises treating a scar. According to yet another embodiment, treating a scar comprises reducing scar collagen abundance, scar width, and scar tissue contracture and decreasing and preventing scar formation, and scar reduction.

The term “skin integrity” as used herein refers to intact skin, which is the body's first line of defense against the invasion of microorganisms, which provides a protective barrier from numerous environmental threats, and facilitates retention of moisture.

The term “impaired skin integrity” as used herein refers to an alteration in the epidermis and/or dermis so that the skin is damaged, vulnerable to injury or unable to heal normally.

The term “scar” or “scar tissue” as used herein refers to fibrous tissue that, as a result of the biological process of wound repair, replaces normal tissue destroyed by injury or disease. The term includes both normal scars formed during tissue repair as well as pathological scars. The types of scars include, but are not limited to, atrophic, hypertrophic and keloidal scars, as well as scar contractures.

Atrophic scars are flat and depressed below the surrounding skin as a valley or hole.

The term “hypertrophic scar” refers to a scar with collagen overproduction that causes the scar to be raised above the surrounding skin. In some embodiments, the scar is a keloid scar. The term “keloid scar” refers to a more serious form of excessive scarring that can grow indefinitely into large, tumorous (although benign) neoplasms. As used herein, the terms “scar tissue contracture” and “scar contractures” refers to a tightening of the skin on or around the scar. Scar tissue contracture occurs as a result of contractile wound healing processes that occur in a scar that has already been re-epithelialized and adequately healed. In some embodiments, scar contracture occurs after a second or third degree bum. According to other embodiments, the scar a hypertrophic scar.

As used herein, the terms, “decreasing scar formation”, “decreasing formation of scar tissue”, “inhibiting scar formation”, “inhibition of formation of scar tissue”, “inhibition of scar formation” and like terms are used interchangeably herein and refer to a prophylactic effect on scar formation such that the tendency of the wound to form a scar is decreased or otherwise reduced compared to an untreated wound. As understood herein, the terms encompass prophylactic effects on the formation of excessive fibrotic scar tissue characteristic of pathological scars. As contemplated herein, the terms encompass any amount of prevention, reduction or amelioration in the incidence, degree, or severity of scar formation which produces a cosmetically more desirable scar, e.g., a scar which is less noticeable in the skin. In this regard, techniques for assessing and grading the severity of scar formation, and patient satisfaction regarding same, are familiar to one of skill in the art, and include various objective methodologies including, e.g., visual assessment and physical palpation of the scar tissue.

The term “scar reduction” and “scar tissue reduction” as used herein refers to an improvement in the aesthetic result and/or a reduction in abnormal tissue caused by wound healing compared to a wound that has not been treated.

The term “improved wound healing” when used herein, refers to improved wound healing compared to wound healing that can be seen in an untreated wound.

Improving wound healing involves increasing the rate of wound healing, for example, a wound may heal at a rate that is hours, days, or weeks faster than when the wound is not being treated. Improving wound healing may also include reducing scar tissue in a wound that is healing or has healed compared to what is expected when the wound is not treated.

The term “fibrosis” is used to describe fibroblast-mediated wound healing processes in a non-CNS tissue. It refers to the formation or development of excess fibrous connective tissue as a result of injury or inflammation or interference with its blood supply. It may be a consequence of the normal healing response leading to a scar, an abnormal reactive process, or without known or understood causation.

The term “wound” generally refers to both open and closed wounds, as described below. A wound can be further classified as an acute or chronic wound. An acute wound is one that does not have an underlying healing defect and usually occurs secondarily to surgery or trauma in a healthy individual, healing quickly and completely. In contrast, a chronic wound is one that has a loss in tissue integrity, produced by insult or injury that is of extended duration or frequent recurrence. As used herein, the term “skin wound” refers to a break in the skin.

The term “open wound” is usually classified according to the object that caused the wound. This includes burns, incisions, lacerations, abrasions, puncture wounds, penetration wounds, gunshot wounds and the like. Incisions or incised wounds may be caused by a clean, sharp-edged object such as a knife, a razor, or a glass splinter. Incisions involving only the epidermis can be classified as cuts. Lacerations are irregular wounds caused by a blunt impact to soft tissue that lies over hard tissue (such as laceration of the skin covering the skull) or tearing of skin and other tissues (such as caused by childbirth). Lacerations may show bridging, as connective tissue or blood vessels are flattened against the underlying hard surface. Abrasions (grazes) are superficial wounds in which the topmost layer of the skin (the epidermis) is scraped off, and are often caused by a sliding fall onto a rough surface. Puncture wounds may be caused by an object puncturing the skin, such as a nail or needle. Penetration wounds may be caused by an object such as a knife entering the body. Gunshot wounds are caused by a bullet or similar projectile driving into or through the body. As such, there may be two wounds, one at the site of entry and one at the site of exit, which is generally known as a through-and-through.

The term “closed wound” refers to contusions, more commonly known as bruises, caused by blunt force trauma that damages tissue under the skin; hematomas, also called blood tumors, caused by damage to a blood vessel that in turn causes blood to collect under the skin; and crushing injuries, which may be caused by a great or extreme amount of force applied over a long period of time.

A variety of conditions may cause scarring, including surgical wounds, burns, cuts, gunshot, etc. Scars commonly form as a result of facial plastic surgery, which includes, but is not limited to, rhytidectomy, blepharoplasty, rhinoplasty, otoplasty, mentoplasty, face lift, forehead lift, brow lift, facial scar revision, facial scar removal, laser surgery, skin resurfacing, wrinkle treatment, plasma skin regeneration, facial fat grafting, skin tightening, tattoo removal and hair replacement.

According to some embodiments, the connective tissue disease comprises an arthritis.

The term “arthritis” refers to a joint disorder or condition that involves inflammation of one or more joints. The term “arthritis,” as used herein, encompasses a variety of types and subtypes of arthritis of various etiologies and causes, either known or unknown, including, but not limited to, rheumatoid arthritis, osteoarthritis, infectious arthritis, psoriatic arthritis, gouty arthritis, and lupus-related arthritis. According to some embodiments, the composition of the present invention is for treating arthritis. According to one embodiment, the arthritis is rheumatoid arthritis.

The term “rheumatoid arthritis” is a chronic inflammatory disease of unknown cause characterized by polyarthritis. Inflammation initially appears in the synovium surrounding the joint but gradually spreads to the periphery of the cartilage and bone, leading to damage and deformity of the joint. In addition to the joints, rheumatoid arthritis can attack other body parts. The symptoms of rheumatoid arthritis include anemia, dry syndromes, subcutaneous nodules, pulmonary fibrosis, vasculitis and skin ulcers.

According to another aspect, the present invention provides a method for treating scar or preventing scar formation in a subject in need thereof comprising topically administering the composition of the present invention to said subject.

According to another embodiment, the present invention provides a method for treating a connective tissue disease in a subject in need thereof comprising topically administering the composition of the present invention to said subject.

The terms “comprising”, “comprise(s)”, “include(s)”, “having”, “has” and “contain(s),” are used herein interchangeably and have the meaning of “consisting at least in part of'. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of ±10%, or ±5%, ±1%, or even ±0.1% from the specified value.

Having now generally described the invention, the same will be more readily understood through reference to the following examples, which are provided by way of illustration and are not intended to be limiting of the invention.

### EXAMPLES

The objective of the study was to evaluate the anti-inflammatory and wound healing properties and the synergy between CBD, CBG or compositions comprising thereof with plant extract Paeonia laciflora. The safety (Example 1) and the anti-inflammatory activity of the Tested Samples (derived from plant extracts and CBD, CBG and terpens) (Examples 2-3) in RAW 264.7 cells (Mouse monocyte macrophage) were examined in LPS-induced inflammatory model (raw cells) and the secretion of three cytokines was monitored.

Then, the compatibility, anti-inflammatory in RAW cells and wound healing in HaCaT cells properties and synergistic action of the selected combination of CBD oils or compositions comprising thereof and Paeonia lactiflora root extract with or without allantoin was determined (Examples 3-5).

### Example 1. Toxicity Evaluation

### Objective

The aim of this experiment was to determine the highest concentration of Tested Samples tolerated by RAW 264.7 cells (Mouse monocyte macrophage) without causing a reduction in cell viability (i.e. not toxic to the cells).

### Experimental Arrangement

The assay was performed on 15 Tested Samples in triplicates (the Tested Samples are labeled 1-11 and 13-16). RAW 264.7 cells (1.7×10⁵ ¢/ml, by counting) were seeded in 96 well plates containing 1701 μl/well of complete growth medium (see formulation section). The cells were incubated at 37° C. with 5% CO₂ for 24 hr. The medium was aspirated and replaced by LPS-containing medium (25 ng/ml) with or without the Tested Samples at four different concentrations, as described below. In addition, Naive cells (Group 1), vehicle treated cells (Group 2), Stimulated Control (Group 3) and Stimulated Vehicle Control (Group 4) served as negative controls. SDS (Group 5) and Dexamethasone (Group 6) served as positive controls for viability and anti-inflammatory assays, respectively. The blank control group was included in the assay (Group 11), and subtracted from all treatments. The cells were incubated at 37° C. with 5% CO₂ for 24 hr. At the end of incubation, the viability of the cells was measured using the MTT assay. Viability greater than 85% was considered nontoxic.

TABLE 1
Experimental design

| Test Group | Description | Concentration | LPS |
|---|---|---|---|
| 1. | Naive | N/A | − |
| 2. | Vehicle | TBD | − |
| 3. | Positive Control | SDS (0.1%) | − |
| | for Viability | | |
| 4. | Stimulated | N/A | + |
| | Control | | |
| 5. | Stimulated | TBD | + |
| | Vehicle | | |
| 6. | Positive Control | Dexamethasone | + |
| | for Anti- | (10 μM) | |
| | inflammatory | | |
| 1. | Tested Samples | Con. 1 (*) | + |
| 8. | (15) | Conc. 2 (*) | + |
| 9. | | Conc. 3 (*) | + |
| 10 | | Conc. 4 (*) | |
| 11 | Blank | N/A | + |

### Tested Samples

| Tested | |
|---|---|
| Sample | Plant extract |
| 1 | Achillea millefolium |
| 2 | Hamamelis virginiana |
| 3 | Hamamelis virginiana |
| 4 | Uncaria tomentosa (Cat's Claw) (by Carrubba) |
| 5 | Uncaria tomentosa (by Coboisa ) |
| 6 | Paeonia lactiflora root extract |
| 7 | Rehmannia chinensis |
| 8 | Rehmannia chinensis |
| 9 | Coptis chinensis |
| 10 | SOPHORA FLAVENSIS |
| 11 | Ganoderma Lucidum |
| 13 | Polygonum cuspidatum (PCE) |
| 14 | Combination of terpenes |
| 15 | CBD |
| 16 | CBG |

### Tested Samples 1-13 are Plant Extracts

Test Sample 14 is a combination of terpenes as following:
myrcene 19.5-30.5 wt %
terpinolene 12-20 wt %
limonene 12-19 wt %
ocimene 8-13 wt %
β-pinen 4-7 wt %
β-caryophyllene 4-7 wt %
α-pinene 2-4.5 wt %
Y-terpinene 2-4.5 wt %

Tested Samples 6, 7, 9, 10 were purchased as powders. 1 g of each was solubilized in 1 ml 60% glycerol (in sterile DDW) to generate the Stock solution (denoted as 1). Three additional stocks were made at 1:5 (denoted as 2), 1:10 (denoted as 3) and 1:50 (denoted as 4) dilutions (performed also in 60% glycerol and stored at 2-8° c). Then, all solutions were diluted 1:1000 in the cell culture media (working concentration). Thus, the final concentrations were_1-1 mg/ml; _2-0.2 mg/ml; _3-0.1 mg/ml and _4-0.02 mg/ml.

Tested Samples 1-5, 8, 11 and 14 were received as liquids. This was considered the first stock solution (denoted as 1). Three additional stocks were made at 1:5 (denoted as 2), 1:10 (denoted as 3) and 1:50 (denoted as 4) dilutions (performed also in 60% glycerol and stored at 2-8° C.). All solutions were diluted 1:1000 in the cell culture media to obtain final dilution of: _1-1:1,000; _2_1:5,000; _3_1:10,000; and as _4-1:50,000.

Tested Samples 15 and 16 (CBD and CBG, respectively) were solubilized in ethanol to reach the final concentrations of 10 μg/ml, 2, 1 and 0.2. In the amended repetition (second preparation of CBD), CBD concentrations were 0.5, 1, 5, 10 and 25 μg/ml, as indicated in the chart. In combinations of CBD or CBG with other compounds CBD was used in concentration of 0.1 or 0.5 μg/ml and CBG in concentration of 2 μg/ml.

### Results

This phase was aimed to determine the nontoxic range of the Tested Samples. Fifteen (15; marked as 1-11 and 13-16) tested Samples were evaluated in this section (data not shown). SDS, used as the positive control, was toxic and caused a significant reduction in cell viability. Safe concentrations for each test item (TI) was chosen to evaluate the efficacy of the test items in anti-inflammatory cell model.

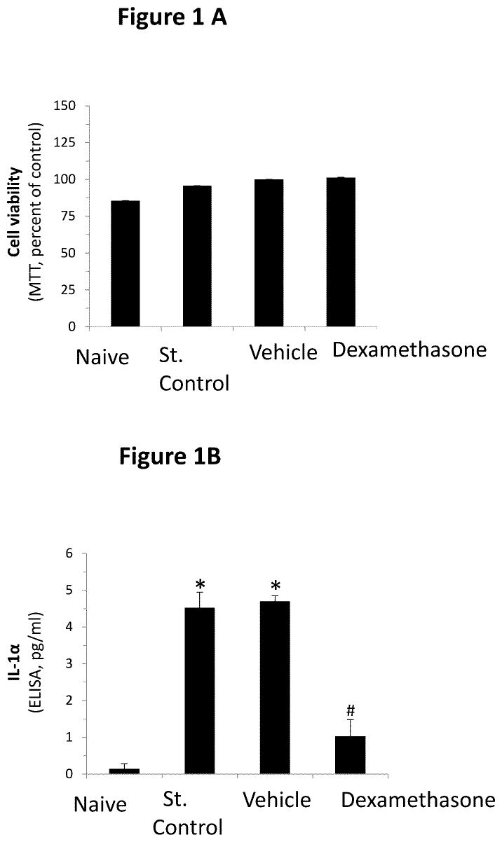

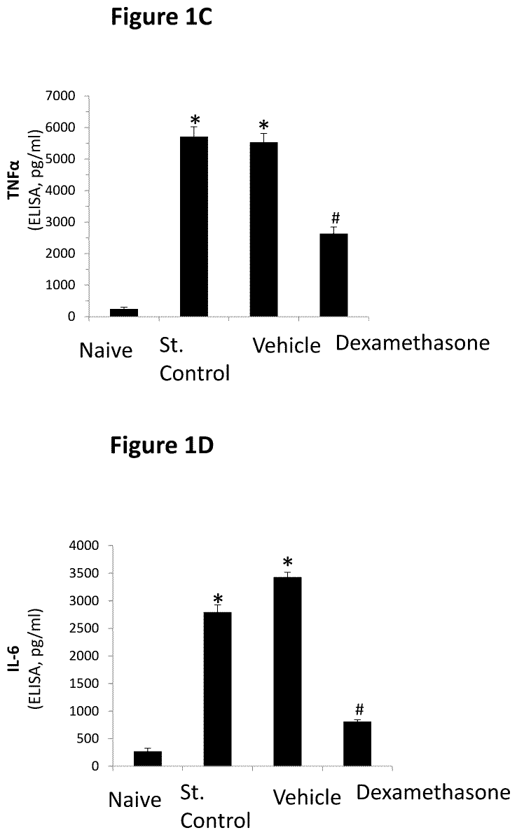

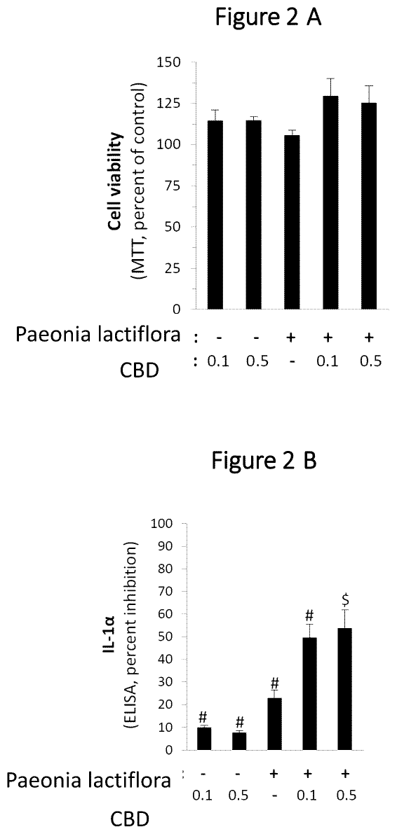

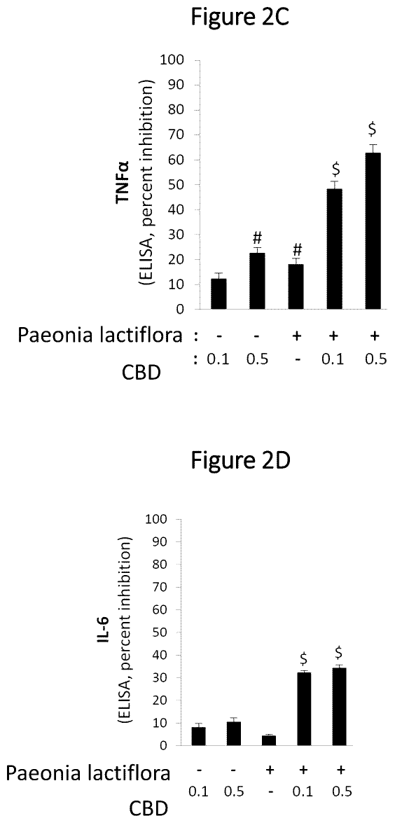

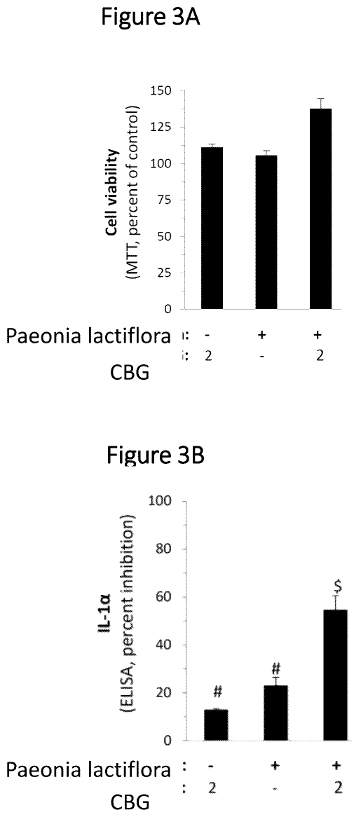

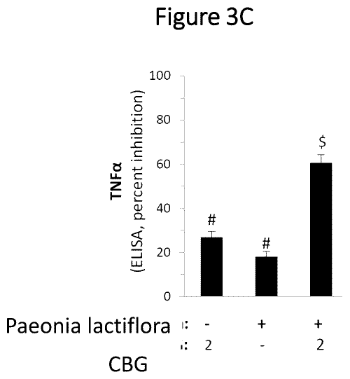

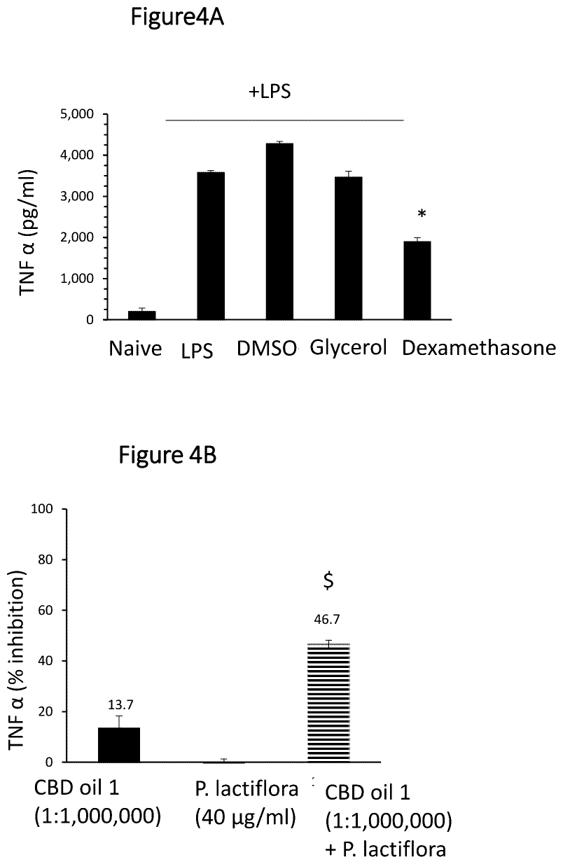

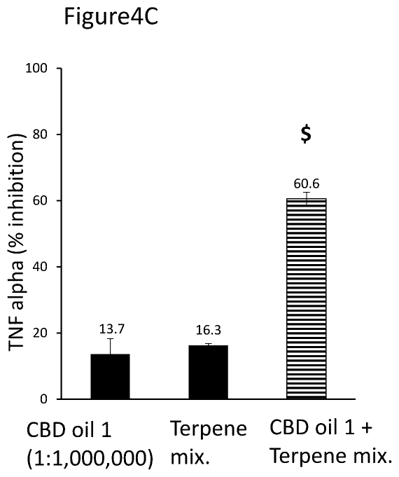

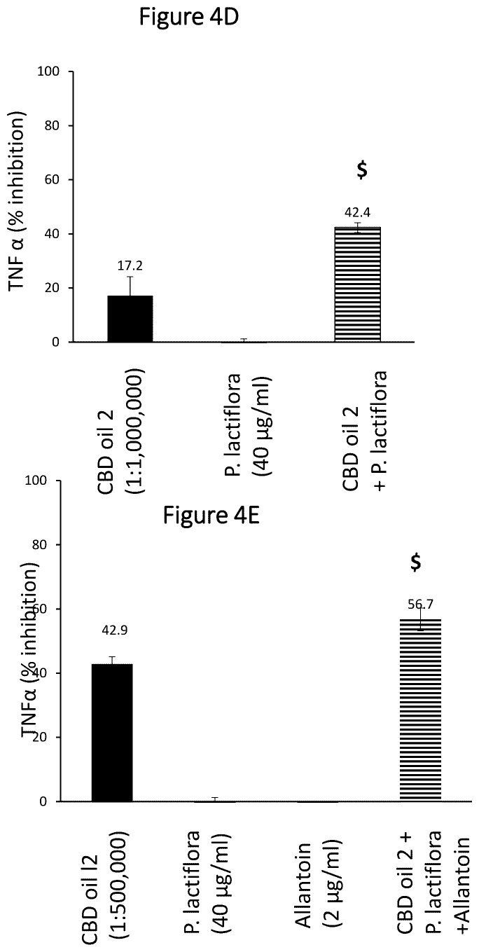

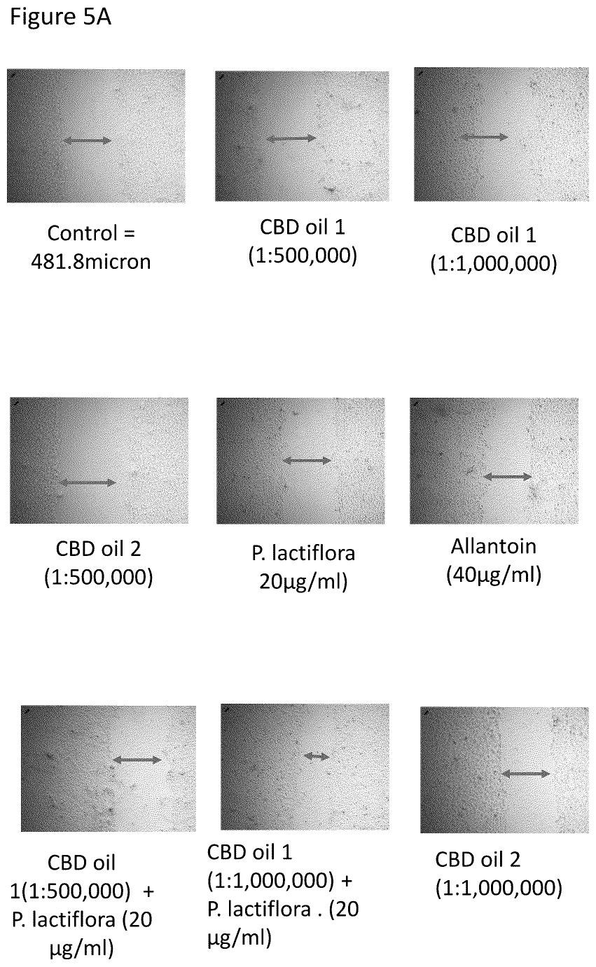

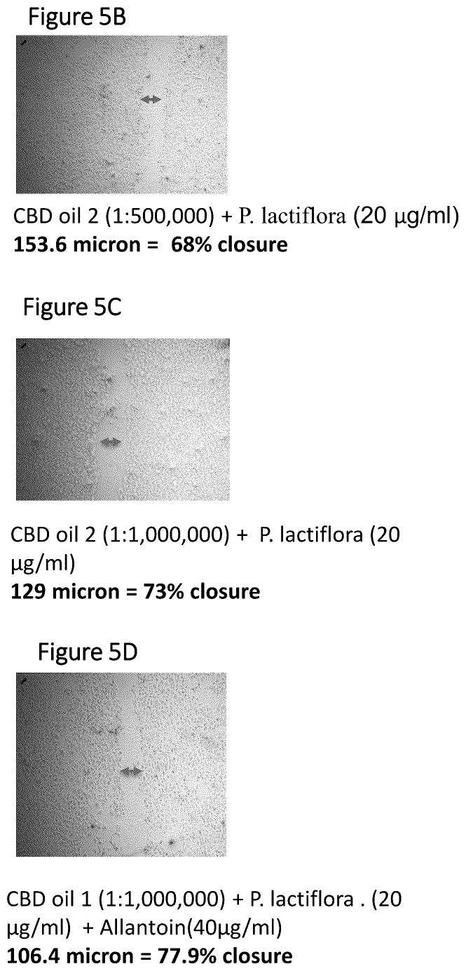

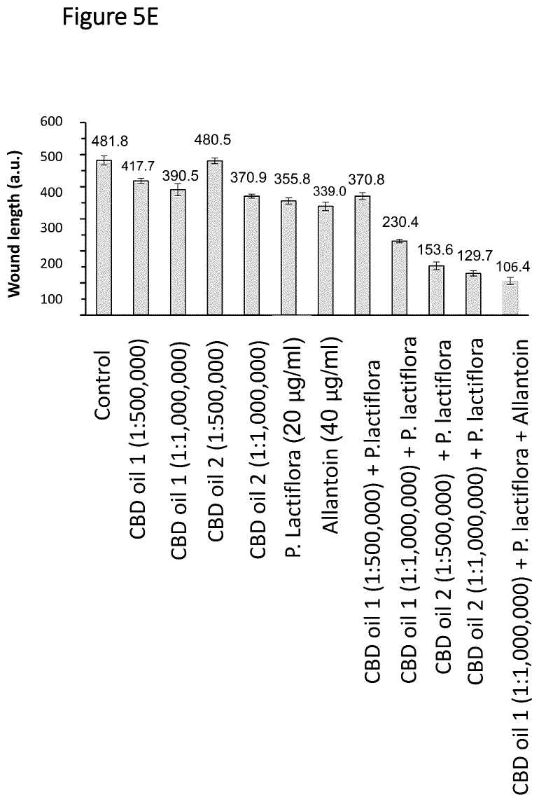

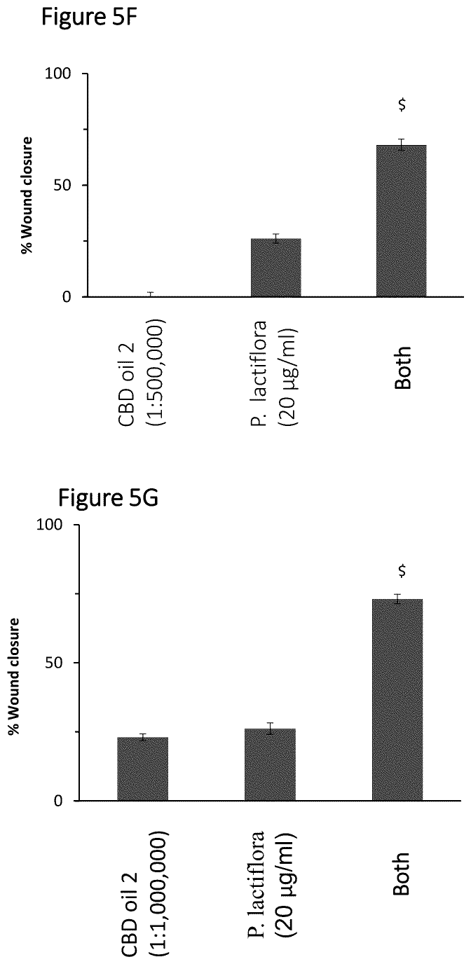

Cell viability of the controls is exhibited in FIG. 1A. Cell viability of CBD, CBG, Paeonia lactiflora herbal extract is presented in FIG. 2A and 3A.

### Example 2. Efficacy Evaluation

### Objective

The aim of this experiment was to determine the anti-inflammatory action of the Tested Samples.

The assay was performed on 15 Test items and was carried out in triplicates (the Tested Samples are labeled 1-11 and 13-16) (Data not shown).

### Experimental Arrangement

RAW 264.7 cells (170×105 ¢/ml, by counting) were seeded in 96 well plates containing 170 μl/well of complete growth medium (See formulation section). The cells were incubated at 37° C. with 5% CO₂ for 24 hr. The medium was aspirated and replaced by LPS-containing medium (25 ng/ml) with or without the Tested Samples at two different concentrations (similarly to what is presented in Table 1). In addition, Naïve cells (Group 1), vehicle treated cells (Group 2), Stimulated Control (Group 3) and Stimulated Vehicle Control (Group 4) served as negative controls. SDS (Group 5) and Dexamethasone (Group 6) served as positive controls for viability and anti-inflammatory assays, respectively. A Blank control group was included in the assay (Group 11), and subtracted from all treatments.

The cells were incubated at 37° C. with 5% CO₂ for 24 hr. At the end of incubation, the viability of the cells was measured using the MTT assay, according to SOP. Viability greater than 85% was considered nontoxic. In addition, the spent media from all test groups was collected under standardized conditions and centrifuged at 250×g for 5 min to remove particulates. Clear supernatants were frozen at −70° C. until cytokine analysis. IL-1α, TNFα and IL-6 levels were measured by ELISA, according to manufacturer instructions. Pro-inflammatory factors, such as interleukin (IL)-1α, IL-1β, IL-6, and tumor necrosis factor-α are upregulated in keloid tissues, which suggests that, in patients with keloids, pro-inflammatory genes in the skin are sensitive to trauma. Keloids and hypertrophic scars are caused by cutaneous injury and irritation, including trauma. This may promote chronic inflammation, which in turn may cause the invasive growth of keloids. In addition, the upregulation of pro-inflammatory factors in pathological scars suggests that keloids and hypertrophic scars are inflammatory disorders of skin, specifically inflammatory disorders.

### Results

As expected, the secretion of all three cytokines (IL-1α, IL-6 and TNFα) were significantly enhanced by LPS. In addition, dexamethasone, a commercial steroid used as positive control, reduced their levels (FIG. 1B, 1C, 1D). Following these tests, combinations of CBD, CBG, terpene mixture and Paeonia lactiflora extract have been assessed to evaluate synergistic activity between the compounds.

### Example 3—Synergistic Anti-Inflammatory Activity Between CBD/CBG with Paenoia lactiflora Plant Extract (P. lactiflora)/Terpene Mixture (FIGS. 1-3).

The objective of the Study was to evaluate the synergistic anti-inflammatory properties of the Tested Samples. First the cell viability test (MTT) was conducted determine the nontoxic range of the Tested Samples and their combination (FIG. 2A and 3A). Viability greater than 85% was considered nontoxic.

Cell culturing and treatments was performed under sterile conditions. Experiments were carried out in a certified RAW 264.7 cells (Mouse monocyte macrophage) obtained originally from ECACC. The cells were incubated at 37° C. with 5% CO₂ for 24 hr. Then, the medium

was aspirated and replaced by LPS-containing medium (25 ng/ml) with or without the Tested Samples. In addition, Naïve cells, vehicle treated cells, Stimulated Control and Stimulated Vehicle Control served as negative controls. Dexamethasone served as positive controls for anti-inflammatory assays. Blank control group was included in the assay, and subtracted from all treatments.

The anti-inflammatory activity of the Tested Samples was examined in the LPS-induced inflammatory model (RAW 264.7 cells) and the secretion of three cytokines was monitored. Then, the compatibility and possible synergistic action of selected combination was determined. IL-1α, TNFα and IL-6 levels were measured by ELISA, according to manufacturer instructions.

Cell viability (FIG. 1A, 2A, 3A) and anti-inflammatory activity of Tested Samples and their combination have been evaluated by inhibition of secretion of IL-1 alpha (FIG. 1B, 2B, 3B), TNF alpha (FIG. 1C, 2C, 3C) and IL-6 (FIG. 1D, 2D).

As expected, the secretion of all three cytokines were significantly enhanced by LPS. In addition, dexamethasone, commercial steroid used as positive control, reduced their levels.

### Anti-Inflammatory Activity of Paeonia Extract (P. lactiflora) with CBD (FIG. 2)

Cell viability test (MTT) was conducted to determine the nontoxic range of the Tested Samples and their combinations (FIG. 2A). Anti-inflammatory activity of combination of CBD with Paenoia extract (Tested Sample 6, P. lactiflora) was evaluated by inhibition of IL-1 alpha (FIG. 2B), TNF alpha (FIG. 2C) and IL-6 (FIG. 2D). CBD was evaluated in two concentrations: 0.1 and 0.5 μg/ml

Paeonia lactiflora (TI6, P. lactiflora) was evaluated at 1000 μg/ml

Paeonia lactiflora extract (Test item 6, P. lactiflora) showed synergistic anti-inflammatory activity with CBD-synergy was found in IL-1α (FIG. 2B), TNFα (FIG. 2C) and IL-6 (FIG. 2D).

IL-1 alpha (FIG. 2B): CBD alone (0.5 μg/ml) showed 7.6% inhibition (0.5 μg/ml), Paeonia extract at 1000 μg/ml (6_1) showed 22.9% inhibition, but together they showed synergistic anti-inflammatory activity of 53.9% IL-1 alpha inhibition.

TNF alpha (FIG. 2C): CBD alone showed 12.3% inhibition (0.1 μg/ml), Paeonia extract (P. lactiflora) at 1000 μg/ml showed 18% inhibition, but together they showed synergistic anti-inflammatory activity by 48.4% TNF alpha inhibition. CBD at 0.5 μg/ml alone showed 22.6% inhibition and together with Paeonia extract (P. lactiflora) at 1000 μg/ml they exhibited synergistic anti-inflammatory activity of 62.8% inhibition. IL-6 (FIG. 2D): CBD alone showed 8.2% inhibition (0.1 μg/ml), Paeonia extract at 1000 μg/ml (P. lactiflora) showed 4.4% inhibition, but together they showed synergistic anti-inflammatory activity of 32.3% IL-6 inhibition. CBD at 0.5 μg/ml alone showed 10.6% inhibition and together with Paeonia extract at 1000 μg/ml (P. lactiflora) they exhibited synergistic anti-inflammatory activity of 34.4% IL-6 inhibition.

### Anti-inflammatory Activity of Paeonia lactiflora (P. lactiflora) Extract with CBG (FIG. 3)

Cell viability test (MTT) was conducted to determine the nontoxic range of the Tested Samples (FIG. 3A). Anti-inflammatory activity of combination of CBG with Paenoia lactiflora extract (Tested Sample 6, P. lactiflora) was evaluated by inhibition of IL-1 alpha (FIG. 3B) and TNF alpha (FIG. 3C).

CBG was evaluated at 2 μg/ml.

Paeonia lactiflora (TI6, P. lactiflora) was evaluated at 1000 μg/ml

The anti-inflammatory activity of the extract and CBG was evaluated as well as their combinations. Paeonia lactiflora extract (Test item 6, P. lactiflora) showed anti-inflammatory synergistic activity with CBG-synergy was found in IL-1α (FIG. 3B) and TNF-α (FIG. 3C).

IL-1 alpha: CBG alone showed 12.9% inhibition (2 μg/ml), Paeonia extract at 1000 μg/ml (P. lactiflora) showed 22.9% inhibition, but together they showed synergistic anti-inflammatory activity of 54.6% IL-1 alpha inhibition.

TNF alpha: CBG alone showed 26.8% inhibition (2 μg/ml), Paeonia extract at 1000 μg/ml (P. lactiflora) showed 18% inhibition, but together they showed synergistic anti-inflammatory activity of 60.5% TNF alpha inhibition.

The above results suggest that synergistic effect was found for the combination of Paeonia lactiflora root extract (P. lactiflora) with CBD and CBG.

The objective of the Study was to evaluate the anti-inflammatory properties of the Tested Samples. First, the anti-inflammatory activity of the Tested Samples was examined in the LPS-induced inflammatory model (raw cells) and the secretion of three cytokines was monitored. Then, the compatibility and possible synergistic action of the selected combination was determined. Considering that the three tested cytokines are related to formation of keloid scar it is assumed that the compositions of the present invention inhibiting secretion of these cytokine may efficiently treat or inhibit formation of scars and in particular keloid scars.

### Example 4: Synergistic Anti-Inflammatory Activity of CBD oils with Paeonia lactiflora (P. lactiflora) Extract (FIG. 4)

The effective combinations of the invention have been retested with two different CBD oils (CBD oil 96% and CBD oil 85% with CBG 1.27%) to detect if the synergistic effect is present also with CBD oils and not just pure CBD as previously detected. The following experiments were conducted with the following Tested Samples:

Two Oils that contain high amount of CBD were tested:
OIL 1 (TI1)
Oil with non-detectable levels of THC. The oil (termed here OIL 1) includes the following components:
Total cannboides-89.6 wt %
Total CBD-85.24 wt %
CBG 1.27 wt %
Terpenes 2% wt %: bisabolol, B-Caryophyllene and Linalool at minimal amounts of (6.9, 3.17 and 1.3 mg/g, respectively).

OIL 2 (TI2)
Oil with non-detectable levels of THC. The oil (termed here OIL 2) includes the following components:
Total cannboides-97.7 wt %
Total CBD-96.06 wt %
CBDV-0.15 wt %
CBG NA
Various terpenes 3 wt %

Experiments were carried out in a certified RAW 264.7 cells (Mouse monocyte macrophage) obtained originally from ECACC. RAW 264.7 cells (approx. 2×105 ¢/ml, by counting) were seeded in 96 well plates containing 170 μl/well of complete growth medium. The cells were incubated at 37° C. with 5% CO₂ for 24. Next, the medium was aspirated and replaced by LPS-containing medium (25 ng/ml) with or without the Tested Samples.

In addition, Naïve cells, vehicle treated cells, Stimulated Control and Stimulated Vehicle Control served as controls (FIG. 4A). Dexamethasone served as positive control for anti-inflammatory assay. A Blank control group was included in the assay

Synergistic anti-inflammatory activity was showed between cannabis oils and Paeonia lactiflora root extract in TNF alpha inhibition:

CBD oil 1 (1:1,000,000 dilution) exhibited 13.7% TNF alpha inhibition alone, Paeonia lactiflora root extract (40 μg/ml) showed no activity alone, but together they exhibited synergistic anti-inflammatory activity of 46.7% TNF alpha inhibition (FIG. 4B). When CBD oil 1 (1:1,000,000 dilution) measured together with terpene mixture (16.3% inhibition alone), synergistic anti-inflammatory activity of 60.6% inhibition of this combination was shown (FIG. 4C).

CBD oil 2 (1:1,000,000 dilution) exhibited 17.2% TNF alpha inhibition alone, herbal extract (40μg/ml) showed no activity alone, but together they exhibited synergistic anti-inflammatory activity of 42.4% TNF alpha inhibition (FIG. 4D).

CBD oil 2 (1:500,000 dilution) exhibited 42.9% TNF alpha inhibition alone, herbal extract (40 μg/ml) and allantoin (2 μg/ml) showed no activity alone, but the three compounds together exhibited synergistic anti-inflammatory activity of 56.7% TNF alpha inhibition (FIG. 4E).

These results show that in addition to the synergistic effect showed between the pure cannabinoids CBD and CBG and Paeonia plant extract, the synergistic effect occur also when using cannabinoids-based oils. As the results indicated similar synergistic activity, it is reasonable to assume that the activity of the CBD/CBG oils is mainly due to direct action of the cannabinoids both pure or as oils. Also, the results suggest that allantoin has additive anti-inflammatory effect when combined with cannabis oil and Paeonia plant extract.

### Example 5. Efficacy of Tested Samples in Wound Healing in HaCaT Keratinocyte Cell Line Model (FIG. 5)

Cell culturing and treatments were performed under sterile conditions. Experiments were carried out in a certified HaCaT cell line (an immortal human keratinocyte line) obtained originally from CLS GmbH.

The aim of this experiment was to determine the ability of the Tested Samples to promote cells to migrate to the artificial wounded area (Scratch Assay). The assay was carried out in triplicates. HaCaT cells (approx. 0.3×106 ¢/mL, by counting) were seeded in 96 well plates containing 200 μL/well of complete growth medium (See formulation section). The cells were incubated at 37° C. with 5% CO₂ until reached 90% confluency (visual estimation, typically—after 24 hr). Then, the medium was replaced with pre-prepared complete medium without or with the Tested Samples (Four concentrations each), at a final volume of 200 μL/well. The following control groups were included in this experiment: Naive cells (Group 1) served as baseline control. Vehicle treated cells (Groups 2) served as negative controls. SDS (Group 3) served as positive control. An additional blank control group was also be included (Group 8). The mean blank control value was subtracted from all measurements. The cells were incubated for 24 hr at 37° C. with 5% CO₂. The viability of the cells was measured using the MTT assay, according to standard operating procedure. Viability greater than 85% (and statistically significant) was considered nontoxic (data not shown).

### Efficacy Evaluation

HaCaT cells are seeded in 24 well plates. After reaching the desire confluency, a wound-like area is created [a gap of approx. 0.4 mm width] and the cells re treated without or with two concentrations [in Triplicated] of the Tested Samples. Wound closure was quantified. In addition, representative images are taken. The cells were incubated for 48 hr at 37° C. with 5% CO₂. Pictures were taken from all wells at the beginning and at the end of incubation to measure the original gap and the reproducibility of the scratch area.

The number of cells migrated to the gap versus treatment was plotted and closure (%) of the area was evaluated.

A model system was used to test the impact of the Tested Samples on wound healing. HaCaT keratinocyte cells were used here. First, to ascertain the non-toxic range, the MTT assay was employed (data not shown).

Next, the scratch assay was employed on the Tested Samples alone and their combinations to test the impact of the Tested Samples on dermal wounds.

The results of the wound healing experiment are shown in FIGS. 5A-5F.

FIGS. 5A-5F exhibit wounds with and without the test samples and their

combinations as photos (FIG. 5A-5D) or graph of wound width (μm) (FIG. 5E).

The results clearly show the superiority of several blends of CBD oils and Paeonia lactiflora root extract over the CBD or CBG compounds or Paeonia plant extract given alone. FIG. 5A presents photos of the cells treated with CBD oils 1 or 2 (according to specification in FIG. 4) Paeonia lactiflora, or allanoin alone or combination of CBD oils with Paeonia lactiflora. Specifically, upper row from left to right: Control 481.8 micron, CBD oil 1 (1:500,000), CBD oil 1 (1:1,000,000); Middle row from left to right: CBD oil 2 (1:500,000); Paeonia lactiflora 20 μg/ml (may also termed here as herbal extract or P. lactiflora); Allantoin (401.1g/ml); Bottom row from left to right: CBD oil 1(1:500,000)+Paeonia lactiflora extract (P. lactiflora, 20 μg/ml); CBD oil 1 (1:1,000,000)+Paoenia lactifloa extract (P. lactiflora, 20 μg/ml); and CBD oil 2 (1:1,000,000).

FIG. 5B to 5D present combinations that were found highly effective in wound closure. Specifically, FIG. 5B presents photo of the cells treated with CBD oil 2 (1:500,000)+Paeonia lactiflora extract (P. lactiflora, 20 μg/ml)=153 micron. This combination demonstrated 68% closure. FIG. 5C presents photo of the cells treated with CBD oil 2 (1:1,000,000)+Paeonia lactiflora extract (P. lactiflora, 20 μg/ml) =129.7 micron. This combination demonstrated 73% closure. FIG. 5D presents photo of the cells treated with CBD oil 1 (1:1,000,000)+Paeonia lactifloa extract (P. lactiflora, 20 μg/ml)+Allantoin (40 μg/ml)-106.4 micron which showed 77.9% closure. FIG. 5E shows graph with bars presenting the closure of the “wound” (“wound length”) showed in FIG. 5A-D with the different treatments. FIGS. 5F and 5G show the significant synergistic wound closure activity of CBD oil 2 at different dilutions: (1:500,000) and (1:1,000,000), respectively with Paeonia lactiflora extract (P. lactiflora, 20 μg/ml) (also termed here herbal extract). Synergy is indicated by $.

The combination of Tested Samples CBD oil 1, Paeonia plant extract (P. lactiflora) and Allantoin had a huge effect on wound healing resulting in more than 77.9% improvement (FIG. 5C).

In addition, the combination of CBD oil 2 at two concentrations tested (at dilution of 1,500,000 or 1,000,000) and with Paeonia extract (P. lactiflora) at 20μg/m1 enhanced wound closure in this system (FIG. 5F and 5G) significantly by 73% and 68% respectively.

Appropriate negative, vehicle and positive control groups are included.

### Example 6. Ex vivo Experiments on Human Skin Tissue

The aim of this phase is to optimize and calibrate the ratios and concentrations of the Tested Samples composing the synergistic proven mixtures in the final developed formulation and to test the safety and efficacy of the final formulations containing the active ingredients mixture/s (CBD and/or CBG and Paeonia lactiflora extract). The final formulation containing different ratio of CBD, plant extract and allantoin will be tested EX VIVO on human skin tissue.

The assays are performed similarly to that have been previously done (inhibition of inflammatory markers) but with skin explant. The markers are MTT and two cytokines (TBD).

### Skin Preparation

The human skin organ culture is obtained from a healthy patient undergoing plastic surgery. The study is initiated at the day of surgery. Fixed size skin explant pieces (0.64 cm2) are cut from the skin tissue, using a designated press apparatus.

The skin pieces are prepared and maintained in air liquid interphase; the explants are laid in 6-well culture plates containing skin culture medium (DMEM supplemented with 100U/ml penicillin and 100 μg/ml streptomycin), dermal side down in the medium and epidermis phasing up. The pieces are left to recover at 37° C. with 5% CO₂. Treatment

After recovery, the skin explant are treated without or with the formulations (3 μl) in the absence or presence of SDS (irritancy causing agent) or LPS, and MTT and/or caspase-3 activity assay is performed according to SOP. After 24 hr, the spent media is collected and replaced with fresh media.

Following the incubation, the epidermis is separated from the dermis. The viability of the epidermis is then evaluated by MTT, according to SOP. Viability greater than 85% is considered as nontoxic.

Concomitantly, the spent medium is collected under standardized conditions and centrifuged at 250×g for 5 min to remove particulates. Clear supernatants is then frozen at −70° C. until cytokine analysis.

Skin inflammation is determined by measuring the secretion level of specific cytokines using commercial ELISA, according to the manufacturer's instructions

Although the present invention has been described herein above by way of preferred embodiments thereof, it can be modified, without departing from the spirit and nature of the subject invention as defined in the appended claims

1-29. (canceled)

30. A topical formulation comprising 0.1 to 5 wt % cannabinoid selected from cannabidiol (CBD) and cannabigerol (CBG) or a composition comprising cannabinoid; 0.2 to 8 wt % plant root extract of Paeonia lactiflora, wherein the extract contains 2-6% paeoniflorin; and optionally 0.2-0.8% allantoin.

31. The formulation of claim 30, the composition comprising cannabinoid in a purity of 81, 82, 83, 84, 85, 86, 87, 88, 89, 90, 91, 92, 93, 94, 95, 96, 97, 98, 99% w/w or more CBD or CBG.

32. The formulation of claim 30, wherein the weight ratio between the cannabinoid and the Paeonia lactiflora extract is from 1:1 to 1:100.

33. The formulation of claim 31, wherein the composition comprising CBD further comprises CBG in a ratio of more than 1:1, 1:10, 1:20, 1:30, 1:40, 1:50, 1:60, 1:70:1:80, 1:90, 1:100 of the CBD:CBG.

34. The formulation of claim 31, wherein the composition comprising CBG further comprises CBD in a ratio of more than 1:10, 1:20, 1:30, 1:40, 1:50, 1:60, 1:70:1:80, 1:90, 1:100 of the CBG:CBD.

35. The formulation of claim 30, wherein the formulation provides a synergistic anti-inflammatory effect as expressed by inhibition of IL-6, TNF alpha or IL-1 alpha.

36. The formulation of claim 30, wherein the formulation provides a synergistic wound-healing effect.

37. The formulation of claim 36, wherein the synergistic wound-healing effect comprises a synergistic anti-scaring effect.

38. The formulation of claim 30, wherein the formulation is a pharmaceutical formulation comprising a pharmaceutically acceptable carrier or the formulation is cosmetic and comprises a cosmetic acceptable carrier.

39. The formulation of claim 30 for use in treating a skin disease, disorder or condition.

40. The formulation for use according to claim 39, wherein the skin disease, disorder or condition comprises a wound.

41. The formulation for use according to claim 40, wherein treating the wound comprises improving wound healing and burn healing.

42. The formulation of claim 39, wherein treating the disease, disorder or condition comprises treating a scar or preventing scar formation.

43. The formulation of claim 42, wherein treating a scar comprises reducing scar collagen abundance, scar width, and scar tissue contracture and decreasing scar formation, preventing scar formation and reducing scar.

44. The formulation of claim 39, wherein the skin condition disorder or condition comprises inflammatory skin condition, atopic dermatitis, psoriasis, seborrhea.

45. A method for treating scar or preventing scar formation in a subject in need thereof comprising topically administering the formulation according to claim 30.
